(19) United States
(12) Patent Application Publication
(10) Pub. No.: US 2020/0289382 A1
(43) Pub. Date: Sep. 17, 2020

(54) EMULSIFYING COMBINATION FOR OBTAINING LOW VISCOSITY WATER-IN-OIL EMULSIONS

(71) Applicant: OLEON NV, Evergem (BE)

(72) Inventor: Hilde PEETERS, Keerbergen (BE)

(21) Appl. No.: 16/646,318

(22) PCT Filed: Sep. 10, 2018
(86) PCT No.: PCT/EP2018/074271

§ 371 Date: Mar. 11, 2020

(30) Foreign Application Priority Data
Sep. 11, 2017 (FR) 1758353

(51) Int. Cl. A61K 8/06; A61K 8/39; A01N 25/06; A23L 29/10

(52) U.S. Cl. CPC A61K 8/062; A23L 29/10; A01N 25/06; A61K 8/39

(57) ABSTRACT

The present invention relates to an emulsifying combination, a composition comprising this combination, and the use of this emulsifying combination in small quantities to obtain a low viscosity water-in-oil emulsion.

The present invention concerns the preparation of an emulsion such as a water-in-oil emulsion and more particularly the use of a MEL as co-emulsifier. The present invention also concerns an emulsifying combination a composition and an comprising it, as well as the preparation processes and uses pertaining thereto.

An emulsion is a dispersion of a liquid in particle or droplet form in another liquid, the two liquids being immiscible with each other. The two immiscible liquids of an emulsion are generally designated “aqueous phase” and “oil phase”. The emulsion according to the invention is more particularly a water-in-oil emulsion thus comprising an aqueous phase dispersed in an oil phase.

More particularly, the emulsions according to the invention have a low dynamic viscosity at 25°, that is to say below 10 Pa·s, preferably below 6 Pa·s. Such emulsions are advantageous for the preparation of sprayable products such as may be found in the cosmetics and food products field.

However, such low viscosity emulsions are unstable.

To stabilize these emulsions, it is known to use silicone oils and/or silicone emulsifiers.

Application WO2003039508 describes water-in-oil emulsions that are fluid and sprayable containing an emulsifier system based on a silicone emulsifier having an HLB less than or equal to 8, an emulsifier having an HLB less than 7 and an emulsifier having an HLB greater than 10, the lipid phase containing an amount less than 25% by weight of silicone oil, preferably that amount is comprised between 2 and 25% by weight, the percentages being expressed relative to the total weight of emulsion.

The stability of a water-in-oil emulsion is all the more difficult to attain when the emulsion comprises a high amount of aqueous phase.

Application US 2002/0102282 A1 describes the obtaining of water-in-oil emulsions comprising at least 70% by weight of aqueous phase relative to the weight of the emulsion, by using an emulsifier selected from polyethers, such as the PEG-45 polymer (dodecyl glycol) and the PEG-22 copolymer (dodecyl glycol). These emulsions have a dynamic viscosity at 25° C. less than 5000 mPa·s.

However, silicone-based substances and PEGs are contraversial. In addition to the fact that they are very persistent, silicone-based substances are not biodegradable, and thus have a negative impact on the environment. PGEs are suspected of making the skin more permeable to foreign bodies which could potentially be dangerous to human health.

There is therefore currently still a need for emulsifier systems to replace these risky substances.

Indeed, whatever the types of industry in which they are used, it is preferable for the emulsifying systems to be environmentally friendly and non-toxic for users. This is particularly important when they are used in the preparation of cosmetic, phytosanitary or food emulsions.

Furthermore, it would be advantageous to develop emulsifier systems having in addition one or more of the following features:
have a good emulsifying capability in a low amount,
enable stable emulsions to be obtained,
enable low viscosity emulsions to be obtained,
enable emulsions to be obtained having a small dispersed particle size, for which obtaining emulsions could be carried out using a process that can be performed at ambient temperature and atmospheric pressure.

The invention thus concerns the use of at least one mannosylerythritol lipid (MEL) as co-emulsifier for polyglycerol polyricinoleate (PGPR).

By co-emulsifier is meant a compound capable of optimizing the emulsion properties (stability, etc.) obtained with an emulsifier.

By “mannosylerythritol lipid” or MEL is meant a molecule comprising a hydrophilic part formed by the mannosylerythritol group, and a hydrophobic part formed by at least one acyl group.

By MEL is more particularly designated a molecule having the following general formula (I):

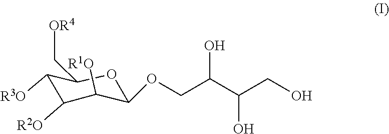

in which:
R¹ and R², which may be identical or different, represent an acly group, comprising an unsaturated or saturated acyclic carbon chain,
R³ and R⁴, which may be identical or different, represent an acetyl group or a hydrogen atom.

Two MEL stereoisomers of formula (I) are known and are represented in the following formulas (II) and (III):

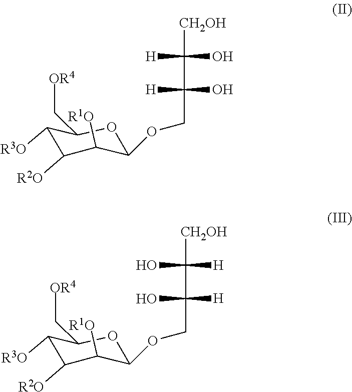

in which R¹, R², R³, R⁴ are identical to those indicated in Formula (I).

Preceding formulas (I) to (III) may represent several molecules, each molecule thus being a MEL. By “MELs” is designated at least two molecules of formulas (I), (II) or (III) that are different by their substitution (acyl groups, acetyl groups) or by their stereoisomerism, more particularly, at least two different molecules of formulas (II).

Moreover, MELs are generally classified into four molecule classes, denoted A to D, according to their degree of acetylation at R³ and R⁴. The MELs-A class comprises molecules of formula (I) having two acetyl groups at R³ and R⁴. The MELs-B class and the MELs-C class comprise molecules of formula (I) having a single acetyl group at R⁴ and R³ respectively. Lastly, the MELs-D comprise molecules of formula (I) not having an acetyl group (R³=R⁴═H).

In addition to varying by their degree of acetylation, MELs may vary in their structure, due to the nature of the fatty acids which compose their hydrophobic part. This variation is generally a function of the process implemented to obtain MELs.

MELs are generally obtained by processes employing fungal culture, and more particularly that of yeasts.

Advantageously the MEL or MELs to which the present application is directed are obtained by a fermentation process, comprising the following steps:
culturing a fungal strain and more particularly a yeast strain in the presence of a carbon source to obtain MELs; and
collecting the MELs so obtained.

The strains from which it is possible to obtain MELs are well-known to the person skilled in the art. By way of example, it is known to use strains of the family Basidiomycetes, preferably of the genus Pseudozyma, such as Pseudozyma antarctica, Pseudozyma parantartica, Pseudozyma aphidis, Pseudozyma rugulosa, Pseudozyma graminicola, Pseudozyma siamensis, Pseudozyma tsukubaensis, Pseudozyma crassa, or of the genus Ustilago, such as Ustilago maydis, Ustilago cynodontis and Ustilago scitaminea.

In general, according to the strain, one class of MELs (MELs-A, MELs-B, MELs-C or MELs-D), is mainly produced, or even exclusively produced, relative to the other MEL classes. By way of example, Pseudozyma antarctica, Pseudozyma aphidis, Pseudozyma rugulosa and Pseudozyma parantarctica produce a majority of MELs-A of formula (III). Pseudozyma graminicola, Pseudozyma siamensis, Pseudozyma hubeiensis produce a majority of MELs-C of formula (III). Pseudozyma tsukubaensis produces a majority of MELs-B of formula (IV) and Pseudozyma crassa produces a majority of MELs-A of formula (IV).

Advantageously, the MELs are obtained by a fragmentation process employing a strain producing MELs of formula (II).

More particularly, the MELs are obtained by a fermentation process employing a strain selected from Pseudozyma aphidis, Pseudozyma rugulosa, Pseudozyma antarctica or Pseudozyma parantarctica, preferably from Pseudozyma aphidis, Pseudozyma antarctica or Pseudozyma parantarctica, more preferably the strain is Pseudozyma aphidis.

The carbon-containing substrate is typically a glycerol, an n-alkane or an oil, in particular of renewable origin.

Any oil, composed of triglycerides and being liquid at the temperature of the fermentation process, may be used as carbon-containing substrate. Preferably, the renewable oil is a vegetable or animal oil, more preferably a vegetable oil. In particular, the vegetable oil is selected from the group consisting of soybean oil, sunflower oil, olive oil and rapeseed oil. More particularly, the vegetable oil is a soybean oil or a rapeseed oil, still more particularly, a rapeseed oil.

These renewable oils are particularly rich in acyl groups comprising a carbon chain with 18 carbon atoms, such as the acyl groups arising from oleic, linoleic and/or linolenic acid.

The fermentation process generally takes at least 3 days, preferably at least 7 days.

According to a preferred embodiment, the MELs are obtained by a fermentation process employing:
a strain of the genus Pseudozyma, preferably Pseudozyma antartica, Pseudozyma parantarctica, or Pseudozyma aphidis,
a vegetable oil, preferably a rapeseed oil or a soybean oil, as carbon-containing substrate.

Such a strain is usually grown in a reactor in a medium comprising glucose, water and/or salts (such as magnesium sulfate, monopotassium phosphate, sodium nitrate, and/or ammonium nitrate). This growth medium is also employed in the fermentation process. As a matter of fact, in general terms, the fermentation medium of the fermentation process comprises a growth medium and the carbon-containing substrate.

Advantageously, the different components of the medium (glucose and strain in particular) are sterilized separately before introduction into the reactor.

The temperature of the medium is preferably comprised between 20° C. and 40° C., more preferably between 25° C. and 35°. It will be noted that, in the context of the present application, and unless otherwise stipulated, the ranges of values indicated are understood to be inclusive.

In the present application, the crude reaction product obtained at the end of the fermentation process is called the crude fermentation product.

The crude fermentation product generally comprises at least two MELs, at least the residual carbon-containing substrate and/or a by-product of the carbon-containing substrate, the strain and water, the by-product of the carbon-containing substrate resulting from the fermentation.

The step of collecting the MELs is directed to separating a MEL or the MELS from one or more of the other components of the crude fermentation product, such as the residual carbon-containing substrate and/or a by-product of the carbon-containing substrate, a strain, and/or water.

According to the above preferred embodiment, the crude fermentation product comprises at least two MELs, at least one triglyceride and/or at least one fatty acid, water and a strain of the genus Pseudozyma.

As a matter of fact, when the carbon-containing substrate is an oil from a renewable source, a by-product of the carbon-containing substrate is a fatty acid. Furthermore, as a vegetable oil is mainly (more than 90% by weight) constituted by triglycerides, the residual vegetable oil is therefore composed of at least one triglyceride.

The separation of a MEL or MELs from one or several of the other components of the crude fermentation product may be carried out by any separation method known to the person skilled in the art.

Advantageously, the separation of a MEL or MELs from one or more of the other components can comprise one or more of the following methods:
decanting,
centrifuging,
filtering,
evaporating,
performing liquid/liquid extraction,
passing over a mineral substrate or a resin.

In particular:
the strain may be separated by decanting, filtering, and/or centrifuging;
the water may be separated by decanting, evaporating, centrifuging, and/or passing over a mineral substrate that is an adsorbent;
the fatty acids and the triglycerides may be separated by performing liquid/liquid extraction and/or by passing over a mineral substrate or a resin.

The collected MELs may thus comprise:
at least one triglyceride and/or at least one fatty acid, and
optionally, a strain.

By “fatty acid” is meant a fatty acid that is free and/or in the form of a salt.

The amount of fatty acid(s) and/or triglyceride(s) present in the collected MELs may be comprised between 0.5 and 60% by weight, preferably between 1 and 50% by weight, relative to the total weight of the collected MELs.

Advantageously, the fatty acid or fatty acids comprise a carbon chain comprising between 8 and 24 carbon atoms, preferably between 8 and 20 carbon atoms.

Advantageously, the triglyceride or triglycerides comprise acyl groups of which the saturated or unsaturated acyclic carbon chain comprises between 8 and 24 carbon atoms, preferably between 16 and 18 carbon atoms. More particularly, the carbon chain is linear and only comprises carbon atoms and hydrogen, possibly substituted by a hydroxyl functional group (OH).

The collected MELs may thus be in a form that is purified to a greater or lesser degree, that is to say in a mixture with other components of the fermentation medium.

More particularly, in the present application, and in particular in the examples, when the collected MELs are in a mixture with at least one fatty acid and at least on triglyceride, optionally water and/or a strain, this mixture is called “MEL mixture”.

A first MEL mixture is a crude fermentation product, that is to say at least two MELs with the other components of the crude fermentation product.

The crude fermentation product may be subjected to one or more separation methods, leading to other preferred MEL mixtures having the following features:
A MEL amount greater than or equal to 30% by weight, preferably greater than or equal to 40% by weight, more preferably greater than or equal to 50% by weight;
an amount of other components (among which fatty acid(s), triglyceride(s), water and/or strain) less than or equal to 70% by weight, preferably less than or equal to 60% by weight, more preferably less than or equal to 50% by weight;

the percentages by weight being given relative to the weight of the MEL mixture.

More particularly, according to the separation method or methods as described above, MEL mixtures of higher or lower MEL concentration may be obtained.

According to a first embodiment, the MEL mixture has the following features:
A MEL amount greater than or equal to 55% by weight;
an amount of other compounds (among which fatty acid(s), triglyceride(s), water and/or strain) less than or equal to 45% by weight;

the percentages by weight being given relative to the weight of the MEL mixture.

Advantageously, in this first embodiment, the amount of water and/or strain is less than or equal to 10% by weight, preferably less than or equal to 5% by weight, relative to the weight of the MEL mixture.

According to a second embodiment, which is particularly preferred, the MEL mixture has the following features:
A MEL amount greater than or equal to 90% by weight, preferably greater than 95% by weight, more preferably greater than or equal to 98% by weight;
an amount of other components (among which fatty acid(s), triglyceride(s), water and/or strain) less than or equal to 10% by weight, preferably less than or equal to 5% by weight, more preferably less than or equal to 2% by weight;

the percentages by weight being given relative to the weight of the MEL mixture.

Advantageously, in this second embodiment, the amount of water and/or of strain is less than or equal to 2% by weight, relative to the weight of the MEL mixture.

Such a MEL mixture may, for example, be obtained using a fermentation process such as described above, comprising several separation steps such as described above, these separation steps preferably including a performing liquid/liquid extraction and/or passing over a mineral substrate.

The passage over a mineral substrate may be chromatography, such as adsorption chromatography with a silica column, carried out using suitable solvents. Such solvents are known to the person skilled in the art.

Examples of mixtures of MELs and of the process for obtaining them are also described in the following publication: “Downstream processing of mannosylerythritol lipids produced by Pseudozyma aphidis”; Rau et al.; European Journal of Lipids Science and Technology (2005), 107, 373-380.

By polyglycerol polyricinoleate (PGPR) is meant a PGPR molecule or a mixture of different PGPR molecules.

As a matter of fact, on account of the many hydroxyl functional groups present in glycerol and polyglycerol, and depending on the process for obtaining the PGPR, numerous reaction products may form and there generally forms a mixture of several PGPR molecules which can differ from each other by:
the number of glycerol units forming the polyglycerol and the arrangement of those units,
the degree of esterification (that is to say the number of esterified hydroxyl function(s) on the polyglycerol), and/or
the number of units of ricinoleic acid forming the polyricinoleate.

Preferably, the PGPR comprises at least one non-esterified hydroxyl.

Preferably, the PGPR mainly comprises (at least 50% by weight with respect to the total weight of PGPR) a polyglycerol polyricinoleate-3 (PG-3-PR) or a polyglycerol polyricinoleate-4 (PG-4-PR). The integer number following the polyglycerol (or PG) represents the number of glycerol units forming the polyglycerol.

Preferably, the PGPR comprises a number of ricinoleic acid units forming the polyricinoleate (PR) comprised between 2 and 12, more preferably between 3 and 10, still more preferably between 4 and 6, in particular, the number of units ricinoleic acid is 5.

Preferably, the hydrophilic-lipophilic balance (HLB) of the PGPR is comprised between 2 and 6.

By HLB (Hydrophilic-Lipophilic Balance) is meant the balance between the size and the strength of the hydrophilic group and the size and strength of the lipophile group of the surfactant. The HLB according to GRIFFIN is defined in J. Soc. Cosm. Chem. 1954 (volume 5), pages 249-256.

PGPR is known to be a good emulsifier for making water-in-oil emulsions.

US2010/0112170 describes the use of PGPR for preparing edible emulsions. However, the final product exemplified in that application is a paste having a plastic structure, and not a liquid emulsion of low viscosity.

Used in a small amount, PGPR alone does not enable a liquid emulsion to be stabilized over time.

By contrast, the use of a MEL as co-emulsifier of the PGPR makes it possible to stabilize a water-in-oil emulsion of low viscosity. This is probably due to the fact that the addition of this co-emulsifier makes it possible to reduce the particle size of the dispersed phase (aqueous phase).

As a matter of fact, the use of MEL is particularly advantageous, since small amounts of MEL and PGPR are then sufficient to obtain a water-in-oil emulsion that is liquid of dynamic viscosity at 25° C. less than 10 Pa·s, preferably less than 6 Pa·s., and stable, that is to say that at ambient temperature, the volume of hydrophobic substances that can appear at the surface of the emulsion is less than 2% by volume relative to the volume of the emulsion throughout the 14 days following the preparation of the emulsion.

Furthermore, the use of MEL as co-emulsifier of PGPR has a synergystic effect. Used alone, MEL does not enable a water-in-oil emulsion to be obtained. The combination of PGPR and MEL makes it possible to obtain a water-in-oil emulsion that is liquid, of low viscosity, and stable, with a lower emulsifier amount than the amount of PGPR required to obtain comparable results.

More particularly, the invention thus concerns an emulsifying combination comprising:
at least one MEL; and
at least one PGPR;
   wherein the MEL/PGPR weight ratio is comprised between 1/99 and 50/50.

The features of the MEL(s) and of the PGPR are as indicated above.

Preferably, the emulsifying combination according to the invention comprises at least two MELs.

The emulsifying combination according to the invention makes it possible to obtain a water-in-oil emulsion with improved properties relative to an emulsion obtained from a PGPR alone.

As a matter of fact, the use of MEL and PGPR enables a water-in-oil emulsion to be obtained with a smaller particle size and of greater stability than an emulsion obtained with PGPR alone.

The emulsifying combination has a good emulsifying capability in a low amount. An amount of at least 0.1% by weight is sufficient to form a water-in-oil emulsion, preferably the amount is at least 0.2%, still more preferably at least 0.3%, the percentages by weight being expressed relative to the weight of emulsion.

The emulsifying combination enables a water-in-oil emulsion to be obtained that is liquid and of low viscosity. By “low viscosity” is meant a dynamic viscosity at 25° C. of less than 10 Pa·s, preferably less than 6 Pa·s.

In particular, the higher the proportion of MEL, the lower the viscosity.

The emulsifying combination enables a stable water-in-oil emulsion to be obtained. By “stable” is meant that at ambient temperature, the volume of hydrophobic substances which may appear at the surface of the emulsion is less than 2% by volume relative to the volume of the emulsion throughout the 14 days following the preparation of the emulsion.

In particular, the 1% by weight of emulsifying combination enables the time of stability of an emulsion to be doubled compared with an identical emulsion prepared with 1% PGPR.

The emulsifying combination enables a water-in-oil emulsion to be obtained with small dispersed particle size.

In particular, the average diameter of the group of particles representing 50% of the volume of the particles (“Dv50”) is less than 20 μm, preferably less than 15 μm, more preferably less than 12 μm.

The emulsifying combination according to the invention may be employed at ambient temperature and atmospheric pressure in particular on preparing a water-in-oil emulsion.

These features of the emulsifying combination according to the invention are more fully described in Example 2 below.

Advantageously, the MEL and the PGPR come from renewable sources and are not toxic for the environment and for users.

Preferably, in the emulsifying combination according to the invention, the MEL(s)/PGPR weight ratio is comprised between 5/95 and 45/55, more preferably between 10/90 and 40/60, still more preferably between 15/85 and 35/65.

Advantageously, the MEL(s)/PGPR weight ratio is comprised between 20/80 and 30/70. Such a ratio makes it possible to further improve the stability of the water-in-oil emulsion obtained with the emulsifying combination according to the invention.

Advantageously, the HLB of the emulsifying combination is comprised between 2 and 6, and preferably between 4 and 6.

Advantageously, the combination comprises at least 50% by weight, preferably at least 60% by weight of PGPR relative to the total weight of the emulsifying combination.

According to a first preferred embodiment of the emulsifying combination according to the invention, it comprises or consists of:
at least 10% by weight at least one MEL; and
at least 60% by weight of PGPR;

in which, the MEL(s)/PGPR weight ratio is comprised between 10/90 and 40/60.

The preferred and advantageous features of the emulsifying combination according to the invention described above apply to this embodiment.

The invention also relates to a process for preparing the emulsifying combination according to the invention, comprises a step of mixing MEL(s) and PGPR, wherein the MEL(s)/PGPR weight ratio is comprised between 1/99 and 50/50.

Advantageously, mixing is carried out at a temperature comprised between 40 and 60° C., preferably between at 60° C.

The MEL(s) and PGPR may be heated independently of each other prior to the mixing or be heated during the mixing.

Advantageously, the emulsifier and the co-emulsifier employed in the process for preparing the emulsifying combination according to the invention have one or more of the preferred features described above.

The emulsifying combination obtained is fluid at ambient temperature and may thus be easily manipulated, in particular for the preparation of compositions and emulsions.

The invention moreover also concerns a composition comprising:
at least one mannosylerythritol lipid (MEL);
a polyglycerol polyricinoleate (PGPR); and
a hydrophobic substance;
   wherein, the MEL(s)/PGPR weight ratio is comprised between 1/99 and 50/50.

The Mel and the PGPR are as described above. In particular, the composition according to the invention comprises at least two MELs.

By “hydrophobic substance” is meant a substance which can be present in the oil phase of an emulsion, with the exception of the emulsifier(s) and co-emulsifier(s) and in particular of the emulsifier and co-emulsifier mentioned in the emulsifying combination according to the invention.

The hydrophobic substance may be selected from the group consisting of mineral oils, silicones, fatty acids, fatty alcohols, esters (other than PGPR), ethoxylated derivatives thereof and mixtures thereof.

More particularly, the mineral oil is liquid paraffin.

More particularly, the silicone is selected from dimethicones and cyclopentasiloxane.

More particularly, the fatty acid is selected from stearic acid, cetylic acid and cetarylic acid.

More particularly, the fatty alcohol is selected from stearic alcohol, cetyl alcohol and cetearyl alcohol.

More particularly, the ester other than PGPR is selected from the group consisting of:
monoesters obtained from a mono-alcohol, such as methanol, ethanol, butanol, isopropanol, octanol, heptanol, ethylhaxanol, stearyl alcohol, isostearyl alcohol, dodecanol, isodecanol, isononanol, isooctanol, and a mono-carboxylic acid, such as caprylic acid, capric acid, heptanoic acid, palmitic acid, myristic acid, lauric acid, isostearic acid and oleic acid.
diesters obtained from a mono-alcohol, as previously described, and from a dicarboxylic acid, such as fumaric acid, adipic acid, succinic acid and sebacic acid;
diesters obtained from monopropylene glycol and a monocarboxylic acid as described above;
the triesters obtained from glycerol and fatty acid(s), and in particular triglycerides;
tetraesters obtained from a tetra-alcohol, such as pentaerythritol, and a mono-carboxylic acid as described above;

and mixtures thereof.

More particularly, the triglyceride comprises acyl groups of which the hydrocarbon chain is linear.

Advantageously, the triglyceride is in the form of a mixture of triglycerides, which comprise saturated linear hydrocarbon chains, in particular chains of 8 to 16 carbon atoms, more particularly chains of 6 to 12 carbon atoms.

Preferably, the mixture of triglycerides is introduced into the composition via a substance selected from the group consisting of vegetable oils, vegetable waxes, vegetable butters, animal oils and fats, a particular fraction of one of the above substances, or mixtures thereof. The particular fraction or substance consists of at least 90% by weight of triglycerides relative to the weight of the particular fraction or substance considered. A particular fraction of one of the above substances is obtained by any method known to the person skilled in the art, in particular by distillation.

More preferably, the triglyceride is introduced into the composition via a vegetable oil, a vegetable wax, a vegetable butter, a particular fraction thereof, or mixtures thereof. Still more preferably, the triglyceride is introduced into the composition via a vegetable oil, or a particular fraction of a vegetable oil.

By way of example of a vegetable oil, there may be cited coconut oil, palm kernel oil, palm oil, olive oil, walnut oil, rapeseed or candula oil, maize oil, soybean oil, sunflower oil, grape oil, linseed oil, camphor oil, apricot kernel oil, avocado oil, macadamia nut oil, and sweet almond oil.

By way of example of a vegetable wax, soy wax may be cited.

By way of example of a vegetable butter, there may be cited shea butter, cocoa butter, mango butter, olive butter and kokum butter.

By way of example of animal fat and oil, there may be cited fish oil, and tallow.

Preferably, the hydrophobic substance is selected from esters, other than PGPR, more preferably from the group of esters described above.

In particular, the hydrophobic substance is selected from diisostearyl succinate, pentaerythritol tetraoctanoate, pentaerythritol tetraisostearate, monopropylene glycol diheptanoate, ethylhexyl laurate, isoamyl laurate, triethylhexanoin and mixtures thereof.

Preferably, the hydrophobic substance is selected from the group consisting of mineral oils, fatty acids, fatty alcohols, esters (other than PGPR), ethoxylated derivatives thereof and mixtures thereof.

Indeed, in the context of a cosmetic application, the hydrophobic substance is not a silicone.

Advantageously, the hydrophobic substance is selected from substances intended to be brought into contact with the surface parts of the animal body, and more particularly, human body.

Advantageously, the hydrophobic substance is edible.

Advantageously, the hydrophobic substance is liquid at ambient temperature and at atmospheric pressure.

Advantageously, the composition comprises at least two hydrophobic substances.

Preferably, the amount of PGPR is at least 0.15% by weight relative to the total weight of the composition.

Preferably, the total amount of MEL(s) and PGPR is at least 0.3% by weight relative to the weight of the composition, more preferably at least 0.5%.

Advantageously, the composition according to the invention further comprises an aqueous phase comprising at least 50% by weight of water, relative to the total weight of aqueous phase.

Preferably, the aqueous phase comprises at least 70% by weight, more preferably at least 80% by weight of water relative to the total weight of the aqueous phase.

Preferably, the amount in aqueous phase is comprised between 10 and 90% by weight, more preferably between 15 and 85% by weight, still more preferably between 20 and 80% by weight relative to the total weight of the composition.

The aqueous phase may further comprise an alcohol.

By way of example of alcohols there may be cited glycerol, ethanol, monopropylene glycol, sorbitol, xylitol, 1,3-butylene glycol, dipropylene glycol, benzyl alcohol, phenoxyethanol and octyldodecanol.

Advantageously, the aqueous phase may further comprise an electrolyte. Electrolytes are known to stabilize a water-in-oil emulsion.

By way of example of electrolytes, magnesium sulfate (MgSO₄) or magnesium chloride (MgCl₂) may be cited.

Preferably, the electrolyte is selected from divalent electrolytes, such as magnesium sulfate.

Advantageously, when the composition comprises the aqueous phase, the latter is in water-in-oil emulsion form.

Advantageously, the combination of MEL(s) and PGPR enables an emulsion to be obtained comprising a large amount of water, that is to say up to 90% water.

Preferably, the composition in emulsion form is liquid. In particular, the emulsion has a dynamic viscosity at 25° C. of less than 10 Pa·s, preferably less than 6 Pa·s.

The invention also concerns a process for preparing a composition comprising a step i) of contacting and mixing at least one MEL, PGPR and a hydrophobic substance.

Advantageously, the MEL(s), the PGPR and the hydrophobic substance used in the process for preparing a composition according to the invention have one or more of the preferred features described above.

Preferably, the mixing step is carried out with stirring.

The mixing may be carried out at ambient temperature, or alternatively, at a temperature comprised between 40 and 60° C., preferably at 60° C.

The MEL(s), the PGPR and the hydrophobic substance may be heated between 40 and 60° C., preferably at 60° C., independently of each other, prior to their contacting or being heated during of mixing.

According to a first embodiment, an emulsifying combination is prepared in advance of the contacting with the hydrophobic substance.

The emulsifying combination can be heated in advance of the contacting with the hydrophobic substance.

According to a second embodiment, the hydrophobic substance is first of all contacted with the MEL(s) and/or the PGPR.

The hydrophobic substance and the MEL(s) and/or the PGPR may be heated in advance prior to the contacting.

The invention also relates to a process for preparing a water-in-oil emulsion comprising the following steps:
i. contacting and mixing at least one MEL, a PGPR and a hydrophobic substance to obtain a composition
ii. adding water to the composition obtained in step i) with stirring.

Advantageously, the MEL(s), the PGPR the hydrophobic substance and the water used in the process for preparing an emulsion according to the invention have one or more of the preferred features described above.

Preferably, the stirring in step ii) is carried out at a speed of at least 6000 rpm, more preferably at least 8000 rpm.

Step ii) may require heating, to be adapted according to the melting temperature of the ingredients of the composition.

Step 1) is as described above (advantageous and preferred features and embodiments).

The compositions and emulsifying combinations according to the invention and in particular the composition in water-in-oil emulsion form, may be used for the preparation of products in various applications.

The invention also concerns the use of an emulsifying combination according to the invention, or of a composition according to the invention, in particular in emulsion form in cosmetic products, plant protection products or food products.

An emulsion comprising the emulsifying combination according to the invention has the advantage of being able to be sprayed in the form of fine droplets of a diameter advantageously less than 20 μm. Thus, the emulsion obtained according to the invention may be used in sprayable products or impregnated wipes.

As a matter of fact, an emulsion with fine droplets makes it possible to uniformly deposit it on a surface, to improve the spreading of that emulsion and allow the active agents contained in the small droplets to penetrate more easily the surface on which they have been deposited.

The invention also concerns to a cosmetic, phytopharmaceutical or food product comprising a composition according to the invention, in particular a composition in emulsion form.

By “cosmetic product” is meant more particularly sun or anti-UV products, self-tanning products, makeup removing products, hair care products, moisturizing sprays, deodorant wipes and cleansing or makeup removing wipes.

The cosmetic product according to the invention may also comprise one or more cosmetic, perfume, pigment, colorant and/or preservative active ingredients.

By “plant protection product” is meant more particularly insect repellents, insecticides, fungicides and herbicides.

The plant protection product according to the invention may also comprise one or more plant protection products, such as an insect repelling, insecticide, fungicide or herbicide active ingredient.

By “food product” is meant more particularly cooking oils and margarines with a reduced fat amount.

The food product according to the invention may also comprise one or more preservatives.

An emulsion comprising the Mel(s) and the PGPR has the advantage of being able to be sprayed in the form of fine droplets of average diameter Dv50 advantageously less than 20 μm, preferably less than 15 μm, more preferably less than 12 μm. Thus, the emulsion obtained according to the invention may be used in sprayable products or impregnated wipes.

Lastly, the invention relates to a sprayable product comprising a reservoir and a system for propelling an emulsion, said emulsion comprising an emulsifying combination according to the invention, or a composition according to the invention.

The invention will be better understood in the light of the following examples, given by way of illustration.

### EXAMPLE 1: MATERIALS AND METHODS EMPLOYED IN THE EXAMPLES

The components used in the following Examples are the following:
emulsifiers and co-emulsifier:
   the mixture of MELs 1B prepared at point 1 below (HLB=9);
   the polyglyceryl-3 polyricinoleate, RADIAMULS POLY 2251K from Oleon (HLB=3.5), (hereinafter designated “PGPR”)
hydrophobic substances (oil phase):
   the capric/caprylic triglycerides, Radia 7104 from Oleon (hereinafter referred to as “MCT”);
   the isoamyl laurate, Jolee 7750 from Oleon
   the propylene glycol diheptanoate, Jolee 7202 from Oleon;
   Rapeseed oil, Lesieur
hydrophilic substances (aqueous phase):
   demineralized water;

The specific material used in the Examples:
an IKA® RW 20 mechanical stirrer;
an Ultra Turrax® T25 IKA-Labortechnik homogenizer;
a Brookfield viscometer;
a Mastersizer 2000 granulometer from Malvern.

1. Obtaining MELs

The MELs were obtained by a fermentation process comprising the following steps:
culturing a yeast strain such as Pseudozyma aphidis in the presence of vegetable oil (rapeseed) to obtain the MELs; and
collecting the MELs so obtained.

Further to the step of collecting the MELs, a first MEL mixture (MEL mixture 1A) is obtained, which has the following features:
MEL amount: 55% by weight
Amount of other components: 45% by weight (of which 42% by weight of triglycerides and free fatty acids and 3% by weight of water and strain),
   the percentages by weight being given relative to the weight of the MEL mixture obtained.

A step of purifying the MEL mixture 1A was then carried out by adsorption chromatography with a silica column, using a mixture of solvents having increasing polarity gradient. A second MEL mixture (MEL mixture 1B) was thus obtained, which has the following features:
MEL amount: at least 98% by weight, relative to the total weight of the MEL mixture obtained.

In particular, each of the mixtures of MELs 1A and 1B comprise MELs-A in an amount of 48% by weight, MELs-B in an amount of 24% by weight, MELs-C in an amount of 27% by weight, and MELs-D in an amount of 1% by weight, the percentages by weight being given relative to the weight of the total amount of MELs.

### Example 2: Preparation of Combinations of Emulsifiers According to the Invention

The MEL mixture 1B and the PGPR are heated to 60° C. and mixed, in the proportions indicated in Table 1 below, using a mechanical stirrer at a speed of 500 rpm until a homogenous mixture is obtained. The different combinations of emulsifiers prepared are summarized in the following Table 1.

TABLE 1
Combinations of emulsifiers according to the invention

| | MEL mixture | PGPR | |
|---|---|---|---|
| | 1B (%) | (%)* | HLB |
| Emulsifying | 10 | 90 | 4.05 |
| combination 1 | | | |
| Emulsifying | 20 | 80 | 4.60 |
| combination 2 | | | |
| Emulsifying | 30 | 70 | 5.15 |
| combination 3 | | | |
| Emulsifying | 40 | 60 | 5.70 |
| combination 4 | | | |

*The percentages indicated are percentages by weight with respect to the total weight of the combination

At ambient temperature and atmospheric pressure, the combinations 1 to 4 are liquid.

### Example 3: Preparation of Compositions and Emulsions According to the Invention

1. Preparation of Compositions

The compositions and emulsions are prepared in the amounts described in Table 2 below. The operating procedures are those described in points 1 and 2 below.

TABLE 2
Compositions prepared in Example 3

| | | Hydrophobic | Aqueous |
|---|---|---|---|
| | Emulsifier | substance | phase |
| Comparative | 1 g PGPR | 30 g MCT | 69 g |
| composition 1 | | | water |
| Composition 1 | 1 g Combination 1 | 30 g MCT | 69 g |
| | emulsifier | | water |
| Composition 2 | 1 g Combination 2 | 30 g MCT | 69 g |
| | emulsifier | | water |
| Composition 3 | 1 g Combination 3 | 30 g MCT | 69 g |
| | emulsifier | | water |
| Composition 4 | 1 g Combination 4 | 30 g MCT | 69 g |
| | emulsifier | | water |

An emulsifying combination according to the invention or a PGPR, and the hydrophobic substance or substances of the oil phase (in the present Example, the MCT) are mixed with mechanical stirring at ambient temperature, i.e. approximately 25° C.+/−5° C., until a homogenous mixture is achieved to respectively obtain a composition according to the invention and a comparative composition.

This process may be implemented if all the hydrophobic substances are liquid at ambient temperature and atmospheric pressure. This is the case for the MCT. In the opposite case, the hydrophobic substance or substances may be heated to a temperature equal to or greater than the highest melting temperature of the substances present before being mixed with the emulsifying combination or PGPR.

2. Preparation of Compositions in Emulsion Form

To the compositions prepared earlier, an aqueous phase is added (69 g in 520 seconds) with mechanical stirring at 800 rpm. Once the addition has terminated, the composition is stirred 1 minute at 10000 rpm using a homogenizer in order to obtain a composition taking the form of a water-in-oil emulsion (hereinafter designated “emulsion” in the Examples).

There are thus obtained emulsions 1 to 4 corresponding to compositions 1 to 4 according to the invention and comparative emulsion 1 corresponding to comparative composition 1.

### Example 4: Evaluation of the Properties of the Emulsions Prepared in Example 3

1. Dynamic Viscosity and Appearance

The dynamic viscosity of emulsions 1 to 4 according to the invention and of comparative emulsion 1 was evaluated one day after their preparation, using a Brookfield viscometer at a temperature of 25° C. and a speed of 10 rpm.

The results are presented in Table 3 below:

TABLE 3
Dynamic viscosity of emulsions 1 to 4 according to the invention and of
comparative emulsion 1 which were prepared in Example 3

| | Dynamic viscosity |
|---|---|
| | (mPa.s) |
| Comparative | 2500 |
| emulsion 1 | |
| Emulsion 1 | 2270 |
| Emulsion 2 | 2190 |
| Emulsion 3 | 1680 |
| Emulsion 4 | 1560 |

The greater the amount of MEL, the lower the viscosity.

The appearance of the different emulsions was evaluated with the naked eye. All the emulsions are white emulsions.

2. Stability

Samples of 15 mL were prepared in graduated 15 mL glass test tubes from the emulsions prepared earlier, and left to stand at ambient temperature.

The stability is observed over time by measuring the volume of hydrophobic substance or substances (MCT in the present Example) that can appear on top of the emulsion. The results obtained are compiled in Table 4 below.

TABLE 4
Stability at ambient temperature of emulsions 1 to 4 according to
the invention and of comparative emulsion 1 prepared in Example 3

| | Volume of hydrophobic substance at the surface (mL) | | | | | |
|---|---|---|---|---|---|---|
| | 1 | 3 | 2 | 1 | 2 | 3 |
| | day | days | weeks | month | months | months |
| Comparative | 0 | <0.1 | <0.4 | 1 | 2 | 3 |
| emulsion 1 | | | | | | |
| Emulsion 1 | 0 | <0.1 | 0.1 | 0.5 | 0.7 | 1.5 |
| Emulsion 2 | 0 | <0.1 | <0.1 | <0.1 | <0.1 | <0.1 |
| Emulsion 3 | 0 | <0.1 | <0.1 | <0.1 | <0.1 | <0.1 |
| Emulsion 4 | 0 | <0.1 | <0.1 | 0.1 | 0.3 | 1.5 |

The results show that by substituting an amount of PGPR by MEL, the stability increases. Emulsions 2 and 3 according to the invention comprise 1% by weight of MELs and PGPR with a MEL/PGPR weight ratio respectively of 20/80 and 30/70, presenting greater stability.

3. Dispersed Particle Size

The measurement of the particle size of the aqueous phase dispersed in the oil phase is measured using the granulometer five days after the preparation of said emulsions.

The average diameter of the particles, volume-weighted, is given according to the following terminology: “Dv50” represents the average diameter of all the particles representing 50% of the volume of the particles.

TABLE 5
dispersed particle size in emulsions 1 to 4 and
comparative emulsion 1

| | Dv50 (μm) |
|---|---|
| Comparative | 17.806 |
| emulsion 1 | |
| Emulsion 1 | 10.309 |
| Emulsion 2 | 6.114 |
| Emulsion 3 | 3.328 |
| Emulsion 4 | 5.109 |

By substituting an amount of PGPR by an amount of MEL, the particle size of the dispersed aqueous phase reduces.

### Example 5: Effects of the Amount of Emulsifying Combination

Emulsions comprising different amounts of emulsifying combination 2 according to the invention prepared in example 2, of MCT and water were prepared, according to the method described in Example 3.

The different emulsifiers prepared are summarized in the following Table 6.

TABLE 6
Emulsions comprising different amounts of emulsifying combination 2
according to the invention

| | Emulsifying | | |
|---|---|---|---|
| | combination | MCT | Water |
| | 2 (%)* | (%)* | (%)* |
| Emulsion 2-0.1 | 0.1 | 30.0 | 69.9 |
| Emulsion 2-0.3 | 0.3 | 30.0 | 69.7 |
| Emulsion 2-0.5 | 0.5 | 30.0 | 69.5 |
| Emulsion 2-0.8 | 0.8 | 30.0 | 69.2 |

*The percentages indicated are percentages by weight with respect to the total weight of the emulsion.

Measurements of dynamic viscosities, stability and evaluation of the appearance were made, in the same manner as in Example 4.

The results as to appearance, viscosity and stability at ambient temperature of the emulsions so prepared are given in Table 7 below.

TABLE 7
Effect of the amount of emulsifying combination on the emulsion

| | | | Volume of | |
|---|---|---|---|---|
| | | | hydrophobic | |
| | Appearance | Dynamic | substance at the | |
| | of the | viscosity | surface (mL) | |
| Emulsion | emulsion | (mPa.s) | 1 day | 3 days |
| Emulsion 2-0.1 | White | 2280 | <0.1 | 2 phases |
| Emulsion 2-0.3 | White | 2220 | 0 | <0.1 |
| Emulsion 2-0.5 | White | 2340 | 0 | <0.1 |
| Emulsion 2-0.8 | White | 1750 | 0 | <0.1 |

Whatever the amount of emulsifying combination, from 0.1% to 0.8% by weight relative to the weight of the emulsion, the emulsions are white and of equivalent dynamic viscosity.

The results show that 0.1% by weight of an emulsifying combination comprising a MELs/PGPR weight ratio of 20/80, relative to the weight of the emulsion, suffices to obtain an emulsion. However, at ambient temperature, such an emulsion is destabilized at the end of 3 days.

More stable emulsions are obtained using at least 0.3% by weight of emulsifying combination 2 according to the invention.

### Example 6: Effects of the Amount of Aqueous Phase

Emulsions comprising different amounts of aqueous phase (demineralized water in the present Example) and oil phase (MCT in the present Example) were prepared using the method described in Example 3 using emulsifying combination 2 according to the invention prepared in Example 2.

The different emulsifiers prepared are summarized in the following Table 8.

TABLE 8
Emulsions comprising 1% of emulsifying combination 2 and
different amounts of aqueous phase and oil phase

| | Emulsifying | | |
|---|---|---|---|
| | combination 2 | MCT | Water |
| | (%)* | (%)* | (%)* |
| Emulsion 2-89 | 1 | 10 | 89 |
| Emulsion 2-79 | 1 | 20 | 79 |
| Emulsion 2-69 | 1 | 30 | 69 |
| Emulsion 2-59 | 1 | 40 | 59 |
| Emulsion 2-49 | 1 | 50 | 49 |

*The percentages indicated are percentages by weight with respect to the total weight of the emulsion.

Measurements of dynamic viscosities, stability and evaluation of the appearance were made, in the same manner as in Example 4.

The results as to appearance, viscosity and stability at ambient temperature of the emulsions so prepared are given in Table 9 below.

TABLE 9
Effect of the amount of emulsifying combination on the emulsion

| | | | Volume hydrophobic | |
|---|---|---|---|---|
| | Appearance | Dynamic | substance at the | |
| | of the | viscosity | surface (mL) | |
| Emulsion | emulsion | (mPa.s) | 8 days | 14 days |
| Emulsion 2-89 | white | — | 0 | <0.1 |
| Emulsion 2-79 | white | 5020 | 0 | <0.1 |
| Emulsion 2-69 | white | 2285 | 0 | <0.1 |
| Emulsion 2-59 | white | 580 | 0 | <0.1 |
| Emulsion 2-49 | white | 250 | 0 | <0.1 |

The greater the amount of water that the water-in-oil emulsion comprises, the higher the viscosity.

At ambient temperature and atmospheric pressure, an emulsion comprising a high amount of dispersed water, up to 89% by weight, remains stable for at least 14 days.

### Example 7: Cosmetic Emulsions

1. Preparation of the Cosmetic Emulsions

The emulsions were prepared in accordance with the amounts, in weight relative to the weight of emulsion, described in Table 10 below. The operating procedure is that described in Example 3, with the exception of the final homogenization, which, in the present example, is for 2 minutes at 9000 rpm.

TABLE 10
Cosmetic emulsions

| | | Emulsion B | Emulsion C |
|---|---|---|---|
| | Comparative | according to | according to |
| | emulsion A | the invention | the invention |
| Emulsifying combination 2 | | 1 | |
| Emulsifying combination 3 | | | 1 |
| PGPR | 1 | | |
| Jolee 7750 | 15 | 15 | 15 |
| Radia 7104 | 8 | 8 | 8 |
| Jolee 7202 | 10 | 10 | 10 |
| Water | 65.3 | 65.3 | 65.3 |
| Preservative | 0.7 | 0.7 | 0.7 |

2. Properties of the Cosmetic Emulsions

Emulsions A, B and C are white.

Measurements of dynamic viscosities, dispersed particle size and stability were made, in the same manner as in Example 4.

The results concerning viscosity, dispersed particle size and stability at ambient temperature of the emulsions so prepared are given in Table 11 below.

TABLE 11
Properties of the cosmetic emulsions

| | | | Volume of hydrophobic | | |
|---|---|---|---|---|---|
| | Dynamic | | substances at the | | |
| | viscosity | Dv50 | surface (mL) | | |
| | (mPa.s) | (μm) | 1 day | 4 days | 7 days |
| Emulsion A | 520 | 16.642 | <0.1 | 1 | 2 |
| Emulsion B | 340 | 2.931 | 0 | <0.1 | <0.1 |
| Emulsion C | 440 | 10.121 | 0 | <0.1 | <0.1 |

Emulsions B and C according to the invention have a dynamic viscosity at 25° C. lower than comparative emulsion A, more particularly less than 500 mPa·s. Emulsion B and C according to the invention moreover have Dv50 particle diameters less than the Dv50 diameter of the comparative emulsion and are more stable at ambient temperature.

Emulsions B and C according to the invention may serve as a base for preparing cosmetic products, in particular sprayable cosmetic products.

### Example 8: Cooking Oils

### 1. Preparation of Emulsions for Use as Cooking Oils

The emulsions were prepared in accordance with the amounts, in weight relative to the weight of emulsion, described in Table 12 below. The operating procedure is that described in Example 3, with the exception of the final homogenization which is for 1 minute at 9000 rpm. The temperature of the emulsions is then brought to 15° C.

TABLE 12
Emulsion E according to the invention and
comparative emulsion E′

| | Emulsion E | |
|---|---|---|
| | according to | Comparative |
| | the invention | emulsion E′ |
| Emulsifying combination 2 | 0.6 | |
| PGPR | | 0.6 |
| Radia 5666 | 79.4 | 79.4 |
| Water | 20 | 20 |

### 2. Properties of the Cooking Oils

2.1. Appearance, Dynamic Viscosity, Average Diameter of the Particles and Stability

The appearance of the two emulsions was evaluated with the naked eye. Emulsion E according to the invention is white whereas comparative emulsion E′ is yellowish.

The dynamic viscosity, the average diameter Dv50 and the stability were evaluated as described in Example 4.

The results are compiled in Table 13 below.

TABLE 13
Properties of emulsion E according to the invention
and comparative emulsion E′

| | | | Volume of hydro- | | |
|---|---|---|---|---|---|
| | Dynamic | | phobic substance at | | |
| | viscosity | Dv50 | the surface (mL) | | |
| | (mPa.s) | (μm) | 1 day | 2 days | 6 days |
| Emulsion E | 130 | 0.361 | 0 | 0 | 0 |
| according to the | | | | | |
| invention | | | | | |
| Comparative | 135 | 7.142 | 0 | 4.5 | 2 |
| emulsion E′ | | | | | phases |

The dynamic viscosity of the two emulsions is comparable.

Using MEL in addition to PGPR makes it possible to reduce the average diameter of the particles representing 50% of the volume of the particles and to stabilize the water-in-oil emulsion.

2.2. Anti-Splash Test

This test is directed to determining the percentage of splashing formed during and after heating the emulsions prepared above.

A pan is heated to 300° C. 20 g of an emulsion at 15° C. is placed in the middle of the pan and a filter paper weighed beforehand is placed over the pan at 6 cm from the hot plate. Once the splashes on the filter paper cease, the latter is again weighed. The difference in weight is equivalent to the mass of cooking oil which splashed from 20 g of cooking oil.

The test is repeated three times. The average splashing rates, calculated for both emulsions are given in Table 14 below.

TABLE 14
Splashing rates of emulsion E according to the invention and
comparative emulsion E′

| | Splashing |
|---|---|
| | RATE (%) |
| Emulsion E | 0.36 |
| Comparative emulsion E′ | 1.58 |

The results show that the use of the emulsifying combination according to the invention makes it possible to reduce the splashing rate by approximately 5 relative to the use of PGPR alone.

This is explained by the fact that the particles in the emulsion according to the invention are smaller and their height of splashing is less relative to the larger particles present in comparative emulsion E′. The small particles not reaching the filter paper situated 6 cm from the pan at the time of the splash, therefore fall back into the pan.

1. (canceled)

2. An emulsifying combination comprising:
at least one mannosylerythritol lipid (MEL), and
at least one polyglycerol polyricinoleate (PGPR),
wherein the MEL/PGPR weight ratio is comprised between 1/99 and 50/50.

3. A process for preparing the emulsifying combination according to claim 2, comprising mixing MEL(s) and PGPR, wherein the MEL(s)/PGPR weight ratio is comprised between 1/99 and 50/50.

4. A composition comprising:
at least one mannosylerythritol lipid (MEL),
a polyglycerol polyricinoleate (PGPR), and
a hydrophobic substance,
wherein the MEL(s)/PGPR weight ratio is comprised between 1/99 and 50/50.

5. The composition according to claim 4, further comprising an aqueous phase comprising at least 50% water.

6. The composition according to claim 5, which is in water-in-oil emulsion form.

7. The composition according to claim 6, wherein the emulsion is liquid.

8. A process for preparing the composition of claim 4, comprising contacting and mixing at least one MEL, PGPR and a hydrophobic substance.

9. A process for preparing a water-in-oil emulsion comprising:
i. contacting and mixing at least one MEL, a PGPR and a hydrophobic substance to obtain a composition, and
ii. adding water to the composition with stirring.

10. (canceled)

11. A sprayable product comprising a reservoir and a system for propelling an emulsion, said emulsion comprising the emulsifying combination according to claim 2.

12. A sprayable product comprising a reservoir and a system for propelling an emulsion, said emulsion comprising the composition of claim 4.

13. A process for preparing a cosmetic product comprising mixing the emulsifying combination of claim 2 with one or more cosmetic, perfume, pigment, colorant and/or preservative active ingredient.

14. A process for preparing a cosmetic product comprising mixing the composition of claim 4 with one or more cosmetic, perfume, pigment, colorant and/or preservative active ingredient.

15. A process for preparing a plant protection product comprising mixing the emulsifying combination of claim 2 with an insect repellant, insecticide, fungicide or herbicide active ingredient.

16. A process for preparing a plant protection product comprising mixing the composition of claim 4 with an insect repellant, insecticide, fungicide or herbicide active ingredient.

17. A process for preparing a food product comprising mixing the emulsifying combination of claim 2 with cooking oil, margarine with a reduced fat amount and/or one or more preservatives.

18. A process for preparing a food product comprising mixing the composition of claim 4 with cooking oil, margarine with a reduced fat amount and/or one or more preservatives.
